(19) United States
(12) Patent Application Publication
(10) Pub. No.: US 2026/0017620 A1
(43) Pub. Date: Jan. 15, 2026

(54) APPOINTMENT SCHEDULING PLATFORM FOR MANAGING A WAITLIST USING INTEGRATED PROGRAMMATIC AND ENGINEERED ARTIFICIAL INTELLIGENCE

(71) Applicant: Ocean Friends, Inc., Irving, TX (US)

(72) Inventors: Animesh Kumar, Irving, TX (US); Manish Shukla, North Bend, WA (US); Kushagra Mittal, Delhi (IN)

(73) Assignee: Ocean Friends, Inc., Irving, TX (US)

(21) Appl. No.: 19/265,468

(22) Filed: Jul. 10, 2025

Related U.S. Application Data
(60) Provisional application No. 63/669,676, filed on Jul. 10, 2024.

(51) Int. Cl. G06Q 10/1093; G16H 40/20

(52) U.S. Cl. CPC G06Q 10/1093; G16H 40/20

(57) ABSTRACT

An appointment scheduling platform manages a user accessible waitlist. A user interface receives a request from a user to book an appointment schedule based on user preferences, and available appointment schedules are identified corresponding to user preferences. Further, requests are collected from the user to be added to the waitlist. An appointment monitoring system tracks the waitlist in case of appointment cancellations by using the user interface that displays the waitlist option along with appointment schedules. A machine learning module prioritizes the users on the waitlist using inputs including user preferences, historical data, and medical practitioner notes to create eligibility for the user. A notification module notifies the user of the eligibility list upon the availability of vacant session slots, and a confirmation module confirms the booking of the appointment schedule after the user submits the user selection.

### CROSS-REFERENCE TO RELATED APPLICATION

This application claims the benefit under 35 U.S.C. § 119(e) and 37 C.F.R. § 1.78 of U.S. Provisional Application No. 63/669,676, which is incorporated by reference in its entirety.

### BACKGROUND

### Technical Field

The present disclosure, in general, relates to the field of electronics, and more specifically, for managing a waitlist in an appointment scheduling platform by prioritizing users based on user preferences and real-time availability of vacant slots.

### Description of Related Art

In the field of healthcare, it is a common practice to schedule appointments well in advance. However, due to unavoidable circumstances, scheduled appointments are subject to cancellation or patients may fail to attend. If not efficiently managed, such occurrences can lead to the inefficient use of medical practitioner's time, resulting in schedules getting converted into unutilized time slots.

In today's healthcare industry, therapy appointment scheduling faces significant challenges, with an average global cancellation rate of 35%. Intensifying this issue is the scarcity of therapists, resulting in wait times ranging from three months to two years. This scarcity intensifies the impact of appointment cancellations, especially as nearly 90% of cancellations occur within a short timeframe due to emergencies involving children with special needs or unforeseen emergencies.

To counteract the impact of cancellations and the absence of patients, many healthcare facilities adopt the strategy of overbooking appointments. This approach, however, comes with its own set of challenges. In situations where an unexpectedly high number of patients attend their appointments, overbooking can lead to unacceptably long waiting times for patients and potentially impose overtime demands on doctors.

Balancing the equation between making appointment decisions and accounting for various costs is a crucial concern for service providers in this domain. These costs include factors such as the expense associated with patient dissatisfaction and loss of goodwill, the costs incurred due to patient waiting time, the idle time of therapists, and any additional expenses incurred through overtime work. Further, the insurance amount is not refunded to the patient if the corresponding session is cancelled and not rescheduled on time. Also, for the cancellation policies of various medical centers, a huge amount is charged to the patient for the therapy, if they cancel it in some pre-defined time interval. This leads to a great loss for the patient since they have to bear the bulky charges of the treatment and it is not even refunded by the insurance companies, since the appointment is cancelled, which may be due to unforeseen circumstances.

In contrast to the traditional appointment scheduling systems where patients are more or less told by the clinic when to come and whom to see or are given limited options on the phone, electronic appointment booking practices make it possible to better accommodate patient preferences by providing patients with more options. Giving patients more flexibility when scheduling their appointments has benefits that can go beyond simply having more satisfied patients. More satisfied patients lead to higher patient retention rates, which potentially allows providers to negotiate better reimbursement rates with payers. More satisfied patients can also lead to reduced absence rates, helping maintain the continuity of care and improve patient health outcomes.

Presently the healthcare industry heavily relies on manual intervention, with administrative staff tasked with contacting users to inform them of appointment cancellations. Users are then required to navigate through available slots and manually book appointments, adding further stress to an already burdensome system. This manual approach not only consumes valuable time of the staff but also leads to inefficiencies and delays in rescheduling, intensifying the challenges faced by both users and therapists.

In recent years, there has been a growing demand for personalized healthcare experiences. Patients seek flexibility in scheduling appointments, and healthcare providers aim to optimize their resources while accommodating patient preferences. Additionally, the advent of digital technologies and online platforms has opened up new avenues for improving the appointment scheduling process.

Scheduling patient appointments in healthcare facilities is a critical aspect of ensuring timely and effective medical care. Traditional appointment scheduling systems often rely on fixed time slots, which may not align with the preferences or constraints of patients. Furthermore, the existing systems may not provide mechanisms for handling appointment cancellations or rescheduling in real-time, leading to inefficiencies and potential delays in patient care.

### SUMMARY

A data processing system and method manage a waitlist in an appointment scheduling platform by prioritizing the user based on the user preferences and real-time availability of vacant slots. The user can opt for the waitlist option if the recommended session slots are not according to the user's preference.

A waitlist management method manages a waitlist in an appointment scheduling platform is disclosed. The method disclosed herein involves receiving a request from a user to book an appointment schedule based on at least one user preferences, and identifying at least one available appointment schedules corresponding to at least one user preferences. Each appointment schedule includes at least one session slots. A waitlist option is presented to the user if at least one user preferences do not match with the available appointment schedules. The user provides at least one waitlist related information via a presented user interface. Based on the provided waitlist related information and cancellation of session slots by other users, at least one preferred vacant session slots are identified in real-time. The user is notified about the availability of at least one preferred vacant session slots, where the notification is sent to the user based on at least one pre-defined criterion.

A computer-implemented data processing system manages a waitlist in an appointment scheduling platform is disclosed. The system includes an appointment scheduling platform that can be accessed by a user using a user device. A user interface integrated within the appointment scheduling platform to receive a request from a user to book an appointment schedule based on at least one user preferences. At least one user preferences include at least one expert's details, timestamps, date of therapy, appointment duration, and so on. At least one available appointment schedules are then identified corresponding to at least one user preferences. Each appointment schedule includes at least one session slots. Further, at least one requests are collected from the user to be added to the waitlist if at least one user preferences do not match with at least one available appointment schedules. An appointment monitoring system tracks the waitlist in case of at least one appointment cancellations by using the user interface that displays the waitlist option along with appointment schedules. The user optionally selects the waitlist option if at least one preferred session slot is not available.

A machine learning module then prioritizes the users on the waitlist based on a plurality of factors based on machine learning algorithms and uses a comparator to match at least one user preferences to create an eligibility of the user The eligibility list is created based upon the exclusion criteria which decides who all users can be excluded from sharing the notifications about the availability of at least one vacant session slots. Finally, a notification module notifies the user of the eligibility list upon the availability of at least one vacant session slots due to at least one cancelled appointments, based on machine learning algorithm-based prioritization of the user and a confirmation module confirms the booking of the appointment schedule after the user submits the user selection.

The appointment monitoring system may further include a contextual analysis module configured to interpret details entered by the user as at least one user preferences. The contextual analysis module operatively coupled to the appointment monitoring system uses LLM (Large Language Model) to interpret details entered by the user as at least one user preferences. The notification shared with the user may include an access link to view and manage their position on the waitlist.

Further, the time duration between at least one notifications shared with the user can be customized by the expert and the user. In addition, the appointment monitoring system may provide multilingual support, allowing the users to make the selections in their preferred language.

### BRIEF DESCRIPTION OF THE DRAWING

The above-mentioned implementations are further described herein concerning the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 depicts an exemplary appointment managing environment for an appointment scheduling platform.

FIG. 2 depicts an exemplary appointment managing process for an appointment scheduling platform.

FIG. 3 depicts an exemplary view of the login page of the appointment scheduling platform.

FIG. 4 depicts an exemplary view of the user interface disclosing a form where users can fill in the details to register for the waitlist.

FIG. 5 depicts an exemplary view of the user interface disclosing a form filled out by the user to register for the waitlist.

FIG. 6 depicts an exemplary view of the user's schedule stored in the appointment scheduling platform.

FIG. 7 depicts an exemplary view of the user interface showing the details of the therapy center chosen as per at least one user preferences.

FIG. 8 depicts an exemplary view of the user interface showing the calendar view of the booked and vacant appointment schedule.

FIG. 9 depicts an exemplary view of the user interface showing the working hours of the expert.

FIG. 10 depicts an exemplary view of the user interface showing at least one preferred slots and vacant session slots in the appointment scheduling platform.

FIG. 11 depicts an exemplary view of the notification shared to the user device when the new session slots are available for booking.

FIG. 12 depicts an exemplary view of the notification i.e., confirmation message shared to the user device, when the appointment scheduled by the user is confirmed.

### DETAILED DESCRIPTION

In the following description, certain specific details are outlined to provide a thorough understanding of various disclosed embodiments. However, one skilled in the relevant art will recognize that embodiments may be practiced without at least one of these specific details, or with other methods, components, materials, etc.

An appointment scheduling platform 102 and appointment monitoring system 112 provide a tangible and, in at least one embodiment, optimal, solution that accounts for various factors that could otherwise lead to an intractable problem. For example, in the medical environment there is often a scarcity of available medical practitioner appointment. Thus, a medical practitioner's time is very valuable and can be critical to a patient. Therefore, the medical practitioner's time should not be underutilized. However, creating a first come, first serve waitlist may be efficient, but this single factor, simplistic approach fails to account for various other factors that if unaccounted for can result in the underutilization of the medical practitioner's time. An appointment managing environment 100 includes an appointment scheduling platform 102 and an appointment monitoring system 112 to schedule users. The appointment scheduling platform and the appointment monitoring system are operatively coupled to each other. A user interface and memory are integrated within the appointment scheduling platform. The appointment monitoring system includes a machine learning module, a comparator, a notification module, and a confirmation module.

The system and method to manage the waitlist includes an appointment scheduling platform 10211 that can be accessed by a user using a user's device. A user interface integrated within the appointment scheduling platform may be utilized to receive a request from a user to book an appointment schedule based on at least one user preferences. At least one user preferences include at least one expert's details, timestamps, date of therapy, appointment duration, and so on. At least one available appointment schedules are then identified corresponding to at least one user preferences. Each appointment schedule includes at least one session slots. Further, at least one requests are collected from the user to be added to the waitlist if at least one user preferences do not match with at least one available appointment schedules. An appointment monitoring system tracks the waitlist in case of at least one appointment cancellations by using the user interface that displays the waitlist option along with appointment schedules. The user optionally selects the waitlist option if at least one preferred session slot is not available. A machine learning module 114 then prioritizes the users on the waitlist based on a plurality of factors based on machine learning algorithms and uses a comparator 116 to match at least one user preferences to create an eligibility of the user The eligibility list is created based upon the exclusion criteria which decides who all users can be excluded from sharing the notifications about the availability of at least one vacant session slots. Finally, a notification module 118 notifies the user of the eligibility list upon the availability of at least one vacant session slots due to at least one cancelled appointments based on machine learning algorithm-based prioritization of the user and a confirmation module confirms the booking of the appointment schedule after the user submits the user selection.

While the description presented herein makes a specific reference to therapy sessions of a child, it is to be appreciated that the appointment managing environment is also equally applicable to other health conditions or medical conditions for which the user has to schedule an appointment with an expert (a healthcare practitioner or expert). For example, the appointment managing environment may be useful in scheduling an appointment for various expert's details such as:
Therapy Sessions: this may include all kinds of therapies like medical therapies (speech, physical, occupational, behavioral, eye movement, and so on), health-related therapies (diet, weight loss, music, psychiatric, and so on);
Medical Tests: this may include bookings made by the user at any hospital, clinic, or center for getting his/her tests done like blood tests, EEGs, ECG, and so on;
Consultation and Follow-Ups: this may include the normal scheduling made by the user for regular health checkups, doctor consultations, follow and so on;
Others: this may include all other activities for which the user needs to schedule an appointment.

Further, the appointment managing environment 100 is described concerning a child as a patient who is undergoing therapy sessions and the user is the child's parent who is accessing an appointment scheduling platform 102 to schedule or reschedule sessions for the user. It should be noted that the patient can be any person (and not just a child) who needs assistance from an expert like a doctor, therapist, physician, clinician, and other expert's details. Also, in the current example, the user is not only limited to the parent of the child, it can be the caregiver, grandparents, family members, and extended family members of the family who have access to the appointment scheduling platform.

Also, the patient can serve as a user, if he/she is capable enough to handle the appointment scheduling platform.

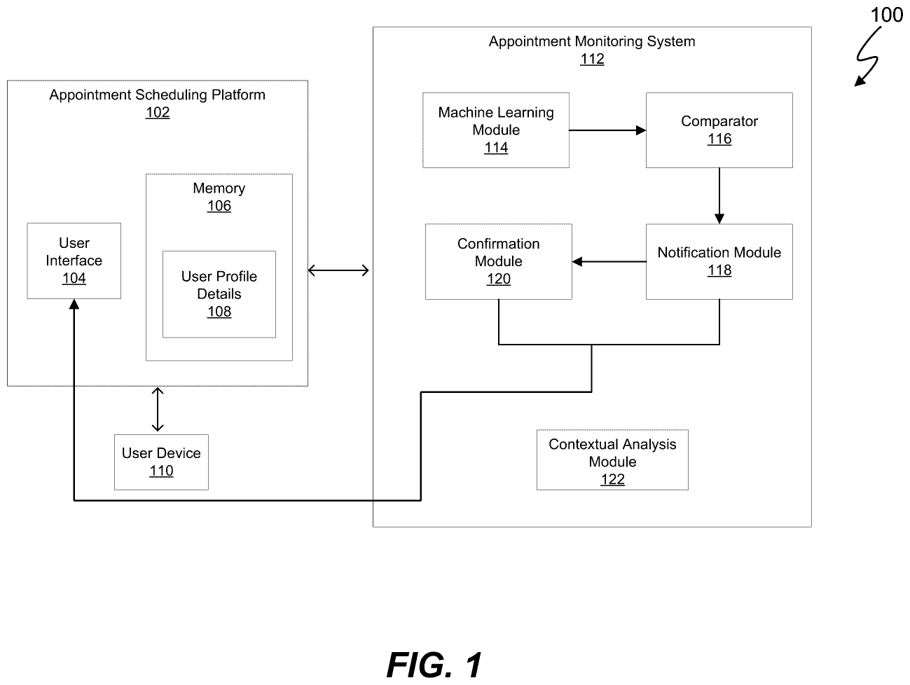

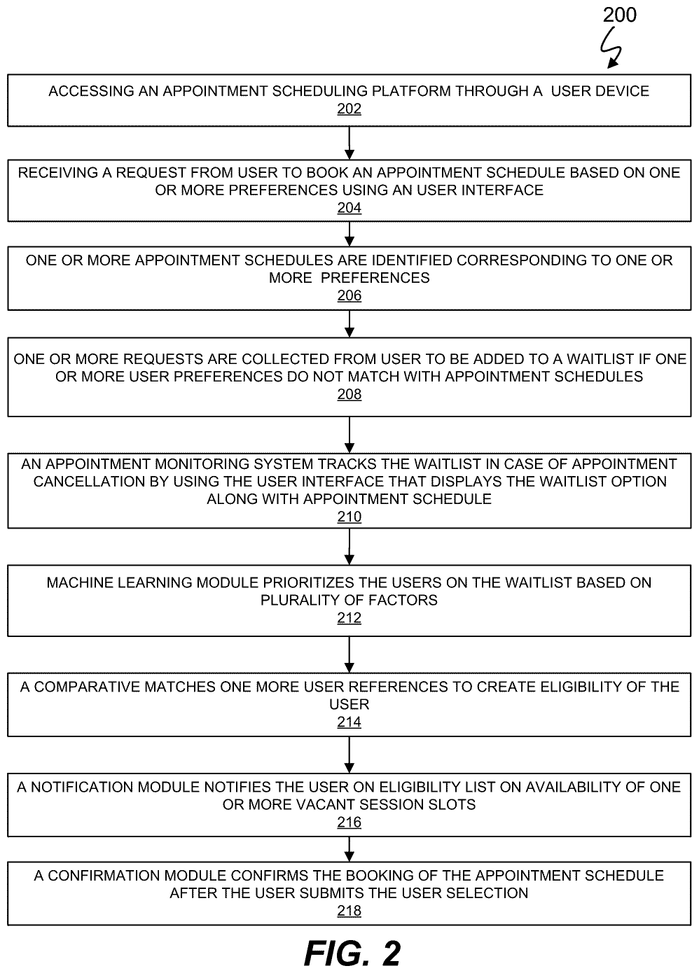

FIG. 1 depicts an exemplary appointment managing environment 100 for an appointment scheduling platform. FIG. 2 depicts an exemplary appointment managing process 200 for an appointment scheduling platform utilized by the appointment managing environment 100.

Referring to FIGS. 1 and 2, in operation 202, an appointment scheduling platform 102 is accessed by a user. The user can access the appointment scheduling platform 102 using his/her device i.e., a user device 110, which may include a mobile, tablet, laptop, computer, or any other similar device. The appointment scheduling platform 102 is operatively coupled to an appointment monitoring system 112.

While using the appointment scheduling platform 102, users interact through their personal devices, denoted as user devices 110. The user device 110 serves as the gateway for users to access the features and functionalities of the appointment scheduling platform 102, allowing them to seamlessly navigate the appointment scheduling process.

Integrated within the appointment scheduling platform 102 is a memory 106 component that stores user preference details 108, ensuring a personalized and efficient scheduling experience. The memory 106 serves as a repository for storing various user-specific information, such as contact details, appointment history, preferred therapists, and any customized preferences. For example, John's user preference in memory 106 of the appointment scheduling platform 102 includes his preferred therapist, Dr. Smith, and his preferred appointment times on weekdays after 6 PM.

When the users access the appointment scheduling platform 102 through the user device 110, the appointment monitoring system 112 retrieves the user preference details 108 from the integrated memory 106, providing a tailored experience based on their preferences and past interactions with the appointment scheduling platform 102. For instance, when Daisy logs in to schedule her next appointment, the appointment scheduling platform 102 automatically displays her preferred therapist and suggests available time slots based on her past scheduling patterns.

In operation 204, a user interface 104 integrated within the appointment scheduling platform 102 receives a request from a user to book an appointment schedule based on at least one user preferences. At least one user preferences include at least one expert's details, timestamps, date of therapy, appointment duration, and so on.

The user interface 104 serves as the primary point of interaction between users and the appointment scheduling platform 102. Integrated seamlessly within the appointment scheduling platform 102, the user interface 104 facilitates the smooth submission of appointment requests based on the user's individual preferences. Typically, the users input various criteria, such as preferred therapists, timestamps, dates of therapy sessions, and desired appointment durations, through the user interface 104. These preferences are crucial for tailoring the scheduling process to meet user's specific needs and preferences. For instance, a user may specify a preference for a particular therapist known for their expertise in a certain therapy type, or they may indicate a preferred time slot based on their availability. By capturing and processing these user preferences, user interface 104 ensures that the appointment scheduling effectively matches users with suitable appointment options. This user-centric approach enhances the overall experience by enabling users to personalize their scheduling requests according to their unique requirements. Additionally, the integration of the user interface 104 within the scheduling platform streamlines the booking process, making it intuitive and user-friendly.

In operation 206, at least one available appointment schedules are identified corresponding to at least one preferences. Each appointment schedule includes at least one session slots.

In the appointment monitoring system 112 outlined in the present disclosure, the process 200 begins with the identification of available appointment schedules that match at least one user preferences. These preferences could encompass various criteria such as preferred therapists, appointment times, locations, or specific types of therapy. Once the user inputs their preferences through the user interface 104, the appointment monitoring system 112 utilizes machine learning algorithms to search and identify appointment schedules that meet these criteria. Each identified appointment schedule comprises at least one session slots, representing the available time slots for appointments within that schedule. For example, if a user, John, prefers therapy sessions on weekday evenings with a specific therapist, the appointment monitoring system 112 will search for appointment schedules that align with John's preferences. It may find multiple available schedules, each offering different session slots on weekday evenings. The appointment monitoring system 112 then presents these options to John, allowing him to choose the appointment slot that best suits his schedule and preferences. By identifying appointment schedules corresponding to user preferences and offering multiple session slots for selection, the appointment monitoring system 112 ensures a tailored and flexible scheduling experience, ultimately enhancing user satisfaction and engagement with the appointment scheduling platform 102.

In operation 208, at least one requests are collected from the user to be added to a waitlist if at least one user preferences do not match with at least one available appointment schedules.

In the waitlist managing environment 100, process 200 of managing appointment scheduling includes a mechanism for handling situations where a user's preferences cannot be immediately accommodated due to the unavailability of suitable appointment slots. When the user attempts to book an appointment and finds that their desired preferences, such as a specific time, therapist, or location, are not available, the appointment monitoring system 112 collects the user's request to join a waitlist. This waitlist serves as a backup option for users, allowing them to indicate their willingness to be notified if a suitable appointment slot becomes available in the future, either due to cancellations or newly opened slots. By collecting these requests and managing the waitlist effectively, the appointment monitoring system 112 ensures that users are provided with alternative options to accommodate their preferences, thereby enhancing user satisfaction and optimizing appointment scheduling efficiency.

For example, Emily, seeking appointments with preferred specialists during specific time slots, may find those slots fully booked. When Emily attempts to schedule a morning appointment due to work constraints but finds none available, the appointment monitoring system 112 collects her request to be added to the waitlist for morning slots. Similarly, James, needing an urgent consultation with a specific physician, joins the waitlist when immediate appointments are unavailable. This feature ensures users are notified of openings that align with their preferences, optimizing scheduling efficiency and user satisfaction.

In operation 210, an appointment monitoring system 112 tracks the waitlist in case of at least one appointment cancellations by using the user interface that displays the waitlist option along with appointment schedules. The user can optionally select the waitlist option if at least one preferred session slot is not available.

The appointment monitoring system 112 manages the waitlist, particularly in response to appointment cancellations. The appointment monitoring system 112 provides users with visibility into both available appointment schedules and the option to join the waitlist if their preferred session slots are unavailable. This allows the users to proactively explore alternative scheduling options and ensures transparency in the process. If the user finds that his desired session slot is not currently available, they can choose to opt for the waitlist, indicating their willingness to be notified in case of cancellations or newly available slots. This user-friendly interface 104 adopts a seamless and interactive scheduling experience, allowing users to make informed decisions and adapt their preferences based on real-time availability of session slots. Using the appointment monitoring system 112 to track the waitlist and provide users with relevant options through the user interface 104, the waitlist managing environment 100 enhances user engagement, optimizes appointment utilization, and ultimately improves the overall efficiency of the scheduling process.

In operation 212, a machine learning module 114 is specially guided and constrained to prioritize the users on the waitlist based on a plurality of factors. The plurality of factors for prioritizing the users on the waitlist includes the duration of the wait, appointment type (one-time or recurring), therapy type, insurance eligibility, and user responsiveness.

In at least one embodiment, at least one artificial intelligence (AI) engines, such as machine learning module 114, serves as a central component of the appointment monitoring system 112. The machine learning module 114 utilizes sophisticated algorithms to analyze a multitude of factors and prioritize users on the waitlist accordingly. By leveraging specially engineered guidance and constraints, the machine learning module 114 can dynamically adapt its prioritization strategy based on real-time data and user interactions, thereby enhancing the efficiency and effectiveness of the scheduling process.

The appointment scheduling platform 102 and an appointment monitoring system 112 set forth herein address technical issues with generating the desired outputs described herein. Conventionally, manual processes were used to generate the desired outputs and were very tedious and time-consuming. The appointment monitoring system 112 utilizes an automated system that does not merely automate a manual process or use a conventional system in a conventional way. The appointment monitoring system 112 utilize at least one artificial intelligence (AI) engines, such as machine learning module 114, and integrate programmatic process management to technologically guide and constrain the machine learning module 114 to produce the desired outputs in a completely different way than any manual process and different than normal use of programs and AI engines. Utilizing specially engineered guidance and control to direct the machine learning module 114 to solve the problems below presents a technical problem that requires a technical solution. The system and method described herein of utilizing the machine learning module 114 are not simply engaging a computer to carry out conventional mental processes, but rather change how computers (and AI systems, specifically) operate to achieve the generation results that were not previously possible or were substantially inefficient prior to the system and method set forth below. The machine learning module 114 utilizes specific technical guidance, control, and constraints to achieve results that are not otherwise achievable.

Prompts are used to guide and constrain each AI engine. The prompts guide the machine learning module 114 by steering the machine learning module 114 to perform specific tasks within defined constraints. “Guiding” an AI engine refers to providing the AI engine with a general direction or framework to shape the machine learning module 114's behavior or decision-making process. Guiding sets goals or principles. Guiding allows the machine learning module 114 some flexibility to interpret and adapt, much like giving it a compass to navigate rather than a fixed path.

Constraining the machine learning module 114 includes imposing specific, hard limits or rules on what the machine learning module 114 can do. Constraining the machine learning module 114 can also include providing specific input data to not only guide but also constrain the scope of the machine learning module 114's reasoning basis and response. Constraining the machine learning module 114 assists with aligning the AI engine(s) for its(their) intended use.

Conventional, non-engineered AI engine prompting has a variety of technical shortcomings. Without proper guidance and constraints, the machine learning module 114 will not produce the desired output specified as produced by the system and method described herein. Instead, the machine learning module 114 will produce many unusable outputs that are unusable for a variety of reasons including so-called “hallucinations” where the machine learning module 114 presents fabricated information, duplicate outputs, too few outputs, too many outputs, outputs that do not meet desired criteria, and so on. Without special technical guidance, the machine learning module 114 cannot reliably be applied to generate desired outcomes.

The system and method generate decomposed, technically engineered AI prompts to include selected and integral AI engine guidance and constraints. The technically engineered prompts are generated and guided with programmatic, automatic inputs specifically designed to unconventionally guide and constrain an AI engine to produce desired outputs, perform quality control to retain or automatically discard outputs that do not meet guidance and constraints, and make the desired outputs available for use, such as use by computer system applications. In at least one embodiment, the problem to be solved by the integrated programmatic and AI engine system and method is uniquely and unconventionally decomposed, and AI prompts are used to solve the decomposed problem. Furthermore, the programmatic inputs to the decomposed AI prompts provide guidance to meet desired output characteristics.

Determining a number of prompts, the guidance and constraints within each prompt, and data flowing from one AI engine prompt to another, in addition to testing a number of prompts for the decomposed problem, testing within each prompt, and validating a desired quality of outputs becomes an intractable combinatorial problem without technical guidance and constraint of the system and method described herein. Thus, the appointment monitoring system 112 implements an integration of programmatic management over decomposed prompts with engineered AI engine guidance and constraints to affect an improvement in AI, programmatic AI management, and AI integrated with programmatic management technology. The appointment monitoring system 112 includes programmatic management, at least one AI engines such as machine learning module 114, and at least one data sources to produce the output described herein that previously could not be produced with conventionally prompted AI engines or could only be produced by humans utilizing a completely different, time consuming, and tedious process. The appointment monitoring system 112 improves conventional methods through the use of a programmatic AI engine management system to generate decomposed, technically engineered AI prompts to include selected and integral AI engine guidance and constraints. It is, for example, the incorporation of the programmatic AI engine management system to generate decomposed, technically engineered AI prompts to include generated, integral, and unconventional AI engine guidance and constraints and execution by the at least one AI engines to provide useful results that improve existing technical processes, which is not an automation of a conventional process.

Programmatic components and AI engines generally utilize at least one processors that have access to memory, which may include at least one storage components, to execute and perform functions. An AI engine is a core hardware and software system that enables artificial intelligence applications to process data, learn patterns, and generate insights or actions. It functions as the brain behind AI-driven systems, facilitating tasks such as machine learning, natural language processing, and decision-making. Exemplary components of an AI engine are:
1. Machine Learning Models—Algorithms that analyze data, recognize patterns, and make predictions.
2. Neural Networks—Deep learning architectures that mimic the human brain for tasks like image and speech recognition.
3. Data Processing Module—Handles raw data input, transformation, and feature extraction.
4. Inference Engine—Applies trained models to make real-time decisions based on new data.
5. Optimization Algorithms—Improves model efficiency, reducing errors and improving predictions.
6. Natural Language Processing (NLP) Module—Enables AI engines to understand, interpret, and generate human language (e.g., chatbots, voice assistants).
7. Computer Vision Module—Allows AI to interpret and analyze images or videos.
8. Reinforcement Learning Mechanism—Helps AI learn from trial and error, optimizing performance over time.
9. API Interface—Connects the AI engine with applications, enabling integration with other software or platforms.

Examples of AI Engines include: XAI's Grok and variations thereof, Google TensorFlow, Meta's PyTorch, Microsoft Azure AI, OpenAI's ChatGPT and variations thereof, IBM Watson, OpenAI Whisper, Google BERT & T5, Amazon Lex, Anthropic Claude, DeepMind's AlphaCode, Google Vision AI, Meta's DINO & SAM (Segment Anything Model), NVIDIA DeepStream. OpenCV AI Kit, Amazon Polly. Google WaveNet, Deepgram.

The plurality of factors considered by the machine learning module 114 for prioritizing users encompasses various dimensions crucial to ensuring an optimized scheduling experience. For instance, one factor is the duration of the wait, which takes into account how long a user has been on the waitlist. Users who have been waiting for an extended period may be given higher priority to ensure fairness and reduce wait times.

Additionally, the appointment type is another factor, and in at least some embodiments is a central factor, in determining prioritization. For example, a user requesting a one-time appointment for urgent care may be prioritized over someone requesting a recurring appointment for routine check-ups. Furthermore, the type of therapy sought by the user is considered, as different therapies may have varying levels of urgency or importance. For instance, a user seeking therapy for a severe mental health issue may be prioritized over someone seeking general counselling services. Insurance eligibility is another crucial factor, as the users with insurance coverage may have different scheduling needs and requirements compared to those without insurance. Prioritizing the users based on insurance eligibility helps ensure that appointments are scheduled efficiently and that users can utilize their insurance benefits effectively. Lastly, the user responsiveness is factored into the prioritization process. The users who are more responsive and proactive in confirming appointments or providing feedback may be given higher priority on the waitlist, as they are more likely to benefit from available slots.

For instance, Alex and John, both find themselves on the waitlist for therapy appointments, but their circumstances and needs differ significantly. Alex has been waiting for two weeks, seeking recurring counselling sessions to address her ongoing anxiety and stress management. Meanwhile, John, who joined the waitlist only three days ago, urgently requires a one-time therapy appointment following a traumatic event. The machine learning module 114, tasked with prioritizing the users on the waitlist, carefully considers several factors. Despite Alex's longer wait time, John's recent request for immediate therapy and acute need for support takes precedence. The machine learning module 114 acknowledges the urgency of John's situation and prioritizes him accordingly, demonstrating how dynamic adaptation and consideration of various factors ensure that users with pressing needs receive timely assistance. This example underscores the ability of the machine learning module 114 to optimize appointment scheduling and enhance user outcomes by tailoring prioritization to individual circumstances.

Below is a specially engineered prompt to guide and constrain the machine learning module 114 to achieve results that are specifically designed to solve scheduling problems within, for example, schedule and priority constraints, utilizing OpenAI's ChatGPT 4.0 as the AI engine of machine learning module 114. Note, in at least one embodiment, the machine learning module 114 generates a prompt and retrieves data from a data source, such as memory 106 and user preference details 108, and utilizes an available application program interface (API) to submit the generated prompt ChatGPT 4.0:

Main Instruction to GPT Model - You are an AI that evaluates candidate reliability
based on attendance history and generates explainable output.

Prompt for GPT -
Prompt = You are an expert assistant predicting which waitlist candidates are
most likely to show up for a scheduled session. ″ \

″Session start date: [programmatically import [session start

date] //, such as ”+ 2025-10-10 10:00:00 + ″, ″ ]//

″Session end date: programmatically import [session end date]

//, such as ″ + 2025-10-10 11:00:00 + ″.\n\n″ \ //

″For each candidate, you are provided with past session count,

cancellation and no-show count, and therapist note and fill-in history. ″ \
import [past session count]
import [cancellation count]
import [no show count]
import [therapist note]
import [fill-in history]

″Rank them from most to least likely to attend the session and

explain your reasoning for each one.\n\n

“Then we add the entry of each waitlist with past session,

cancellation and past fill-in history and the waitlist notes”

″\nReturn a JSON object with two fields: ‘ranking‘ (a list of

waitlistId) and ‘explanations‘ (a dictionary mapping each profileId to a
short explanation”

Following is an embodiment of specally engineered programmatic

integration with artificial intelligence by machine learning module 114 to
specially and programmatically coordinate interfacing of artificial
intelligence of machine learning module 114 and guiding and constraining the
artificial intelligence of machine learning module 114, which can include
internal and/or external AI engines:
defget_show_up_likelihood_order_with_explanation(entries,session_start_date,
session_end_date):

| | |
|---|---|
| # | Build a prompt asking for both ranking and explanation |

prompt = ″You are an expert assistant predicting which waitlist
candidates are most likely to show up for a scheduled session. ″ \

″Session start date: ″ +session_start_date.strftime(″%Y-%m-

%d %H:%M:%S″) + ″, ″ \

″Session end date: ″ +session_end_date.strftime(″%Y-%m-

%d %H:%M:%S″) + ″.\n\n″ \

″For each candidate, you are provided with past session count,

cancellation and no-show count,and therapist note and fill-in history. ″ \

″Rank them from most to least likely to attend the session and

explain your reasoning for each one.\n\n″
for i, e in enumerate(entries):

prompt += f″{i+1}. WaitlistId: {e[′waitlistId′]}, Past

Sessions: {e[′past_sessions′]} , ″ \

f″Cancellations & No

Shows: {e[′cancellations_and_no_shows′]}, ″ \

```
              f″Past Fill-in History: {e[′past_fill_in_history′]}\n″ \
                f″Therapist Note: {e[′notes′]}\n″
```

prompt += ″\nReturn a JSON object with two fields: ‘ranking‘ (a list of
waitlistId) and ‘explanations‘ (a dictionary mapping each profileId to a
short explanation).″

| | |
|---|---|
| # | response = openai.ChatCompletion.create( |
| # | model=″gpt-4″, |
| # | messages=[ |
| # | {″role″: ″system″, ″content″: ″You are an AI that evaluates |

candidate reliability based on attendance history and generates explainable
output.″},

| | |
|---|---|
| # | {″role″: ″user″, ″content″: prompt} |
| # | ] |
| # | ) |

response = client.beta.chat.completions.parse(

```
  model=″gpt-4o-2024-08-06″,
  messages=[
      {
            ″role″: ″system″,
            ″content″: ″You are an AI that evaluates candidate
```

reliability based on attendance history and generates explainable output.″

```
      }, {
            ″role″: ″user″,
            ″content″: prompt
      }
  ],
  response_format=ExtractionSchema
```

```
  )
  gpt_response = response.choices[0].message.parsed
  extracted = gpt_response.model_dump( )
```

| | |
|---|---|
| # | print(extracted) |

return extracted[″ranking″], extracted[″explanations″]

The above prompt act as the input that includes a set of instructions provided to the AI engine, such as the machine learning module 114, so that it can perform a specific function. The structured and informative prompt enables the machine learning module 114 to understand the nuances of the user's request and the context. The prompt is generated via the machine learning module 114. In at least one embodiment, the machine learning module 114 may utilize a prompt generator to generate the prompt. Typically, the prompt generator is a tool, software module, function, or algorithm responsible for taking input and converting it into a usable prompt. The prompt generator is the medium or mechanism through which the prompt can be generated. In at least one embodiment, the prompt generator may be implemented in programming languages such as Python, JavaScript, or Java. In another embodiment, the prompt generator may involve templates, logic structures, predefined keywords, or sentence builders to ensure consistency and clarity in the generation of the prompt. In at least one embodiment, the prompt is generated by the prompt engineer. In at least another embodiment, the skeleton of the prompt (such as a prompt schema) is prepared by the prompt engineer, and then the skeleton is provided to the prompt generator to generate the prompt

The above prompt includes the criteria utilized which decides who should be excluded from receiving notifications about available session slots. The prompt involves incorporating a session start date and a session end date. The session start date and end date define the time window within which the therapy appointment is to be scheduled. The session start date indicates the possible date the user can consider for the therapy appointment, while the session end date specifies the latest possible date. The prompt includes the user's request to appoint a schedule for the therapy in the waitlist. The user request to appoint a schedule include preferred days of the week, times of day, or specific therapists they wish to see during filling for waitlist.

The above prompt also includes the user's historical attendance data. The user historical attendance data includes the count of past sessions attended, the number of cancellations, and the number of no-shows. By incorporating this data, the AI engine gains insight into the user's reliability and behavioral patterns before proving the notification for filling the vacant slot. For example, a user with a high number of no-shows might be scheduled differently. Conversely, a user with a strong record of attendance might be prioritized. This allows the AI engine to personalize and manage appointment availability of at least one vacant session slots due to at least one cancelled appointment.

The fill-in appointments typically refer to those therapy appointments scheduled on short notice, often due to cancellations. The prior fill-in appointment history helps the AI engine understand how often the user has taken advantage of such opportunities in the past. If a user frequently accepts fill-in appointments, they might receive notification of the availability of at least one preferred vacant session slot.

The set of therapist-generated notes are the notes generated by the therapist and are also provided in the prompt. The set of therapist-generated notes contains qualitative data about the user's preferences, therapeutic goals, challenges faced during past sessions, or even interpersonal dynamics that could affect scheduling decisions. For example, a therapist's note may mention that the user prefers morning sessions due to work obligations or that they respond better to biweekly sessions rather than weekly ones. Including therapist notes helps the machine learning module 114 to make decisions. Additionally, the prompt includes optional metadata, such as waitlist notes provided by the user. The metadata refers to supplementary information that may not be strictly required for scheduling but can offer helpful context. For example, a user might leave a waitlist note indicating their flexibility on certain dates, their willingness to take late cancellations, or their preference for a specific therapist or session type. The AI engine, such as machine learning module 114, is guided and constrained based on the prompt.

In operation 214, comparator 116 matches at least one user preferences to create eligibility of the user. The eligibility list is created based upon the exclusion criteria which decides who all users can be excluded from sharing the notifications about the availability of at least one vacant session slots. The exclusion criteria include the exclusion of users who respond late, users whose cancellation rate is high, users whose insurance payments are not made on time, and so on.

The comparator 116 integrated within the appointment monitoring system 112 shapes the eligibility criteria for users awaiting appointment scheduling. The comparator 116 matches at least one user preferences with at least one available slot to determine eligibility, ensuring that users are notified only when relevant options become available. For instance, suppose a user prefers therapy sessions with a specific expert at a particular time and duration. In that case, comparator 116 cross-references these preferences with the available appointment slots to create an eligibility list tailored to the user's requirements.

Furthermore, the eligibility list is refined based on exclusion criteria, which decides who should be excluded from receiving notifications about available session slots. These criteria are designed to prioritize users who are more likely to benefit from the available appointments while filtering out those who may not be as responsive or reliable. For example, users with a history of late responses, high cancellation rates, or delayed insurance payments may be excluded from receiving notifications or shifted to the low-priority side i.e., the users who are given less preference and the notifications to them are shared at the end. By implementing these exclusion criteria, the appointment monitoring system 112 ensures that notifications are directed toward users who are actively engaged and committed to their appointments, thereby maximizing appointment fulfilment and user satisfaction.

In operation 216, a notification module 118 notifies the user of the eligibility list upon the availability of at least one vacant session slots due to at least one cancelled appointment based on machine learning algorithm-based prioritization of the user. The notification module 118 serves a pivotal role in keeping users informed and engaged throughout the appointment scheduling process. When at least one session slots become available due to cancellations, the notification module 118 promptly notifies users on the eligibility list. Using specially engineered guidance and constraint prompts in combination with programmatic logic operations, the notification module 118 prioritizes the users based on various factors, ensuring that those most likely to benefit from the vacant slots receive timely alerts. These notifications are, in at least one embodiment, comprehensive and provide users with not only information about the available slots but also an access link. This link enables users to conveniently view and manage their position on the waitlist, empowering them with real-time updates and control over their scheduling preferences. The user can click on the same access link anytime and get real-time updated information about the vacant session slots.

The notification shared with the user includes an access link to view and manage their position on the waitlist. Also, the time duration between at least one notification shared with the user can be customized by the expert and the user (i.e., user preferences). Moreover, the notification module 118 offers flexibility by allowing customization of the notification frequency. Both experts and users have the autonomy to tailor the timing and frequency of notifications according to their preferences and scheduling needs. This customization feature ensures that notifications align with the user's individual preferences, including the user's schedules, thereby enhancing the overall user experience. For example, if a user is working personnel and does not wish to receive notifications of the vacant session slots throughout the day, that user may identify certain pre-defined criterion for receiving such notifications. For example, if he just wants to get updates in the morning and one update during the evening, the user can customize the notification frequency by himself so that he receives the notifications of the available session slots during his desired time. Similar is the case with therapists, they can also customize the frequency of notifications to be shared with the user as pre-defined criterion.

The AI engine, such as the machine learning module 114, notifies the user about available sessions if at least one of the preferred sessions (i.e., sessions aligned with the user's prior preferences or behavior) is available. Typically, the users indicate their preferred days, times, or types of appointments either explicitly (e.g., selecting morning slots on weekdays) or implicitly (e.g., consistently showing up to Friday afternoon sessions). By limiting notifications to only those that match user preferences, the AI engine avoids spamming users with irrelevant options. The machine learning module 114 take decisions based on the contextual data tied to each individual user, such as a count of past attended sessions, a count of past cancellations and no-shows, a record of prior fill-in participation, at least one therapist-provided notes, and supplemental waitlist notes associated with the user profile

The count of past attended sessions provides insights into the engagement history. A higher number of attended sessions suggests a reliable user, possibly indicating strong therapeutic progress or motivation to continue treatment. The count of past cancellations and no-shows provides a deeper understanding of commitment and potential scheduling issues. A high cancellation or no-show rate may indicate unpredictability, scheduling conflicts, or disengagement. This data is incorporated into the prompt to allow the machine learning module 114 to make a decision to offer time slots. For instance, if a user consistently misses Monday sessions, the machine learning module 114 might deprioritize offering the notification for scheduling the session on Monday. The record of prior fill-in participation refers to instances where the users' made appointments on short notice, typically filling in for cancellations. This allows the AI engine to identify the user with a history of accepting fill-ins.

The therapist-provided notes add a critical qualitative dimension to the decision-making of the machine learning module 114 for providing the notification to the user. Therapist notes may include subjective observations about the client's scheduling preferences, psychological state, therapeutic needs, or even non-verbal cues noted during past sessions. For example, the therapist might note that the user performs better in morning sessions or that they've expressed a desire to limit sessions to weekdays. The waitlist notes associated with the provide optional metadata that the user may have added, often when joining the waitlist. These notes may include requests such as “Only evenings after 5 PM,” “Prefer therapist A over therapist B,” or “Can do virtual appointments only.” By including this data in the decision-making process, the AI engine ensures that any notification it sends aligns not just with historical behavior but also with explicitly stated user preferences.

The AI engine is guided to follow a set of instructions, guidelines, or constraints provided in the prompt to ensure the outputs are consistent. The AI engine is directed to return information in a certain format. The AI engine is constrained to return a structured JSON output. JSON, or JavaScript Object Notation, is a machine-readable and human-readable format. The AI engine follows a predefined schema, with consistent fields, data types, and hierarchies.

Typically, the ranking of the users by predicted attendance, user historical user behavior, and attendance data is used to estimate how likely the given user is to attend a future session. The AI engine considers various features such as how frequently the user has attended sessions in the past, how often they have canceled or missed appointments, whether they respond to short-notice invitations (fill-ins), and even notes left by therapists. Based on these inputs, the AI engine assigns a probability or score to each user that reflects their likelihood of filing at least one vacant session slot due to at least one cancelled appointment. These users are then ranked from most to least likely to attend, allowing the appointment monitoring system 112 to prioritize the notification about the availability of at least one preferred vacant session slots.

For example, if any user has a canceled appointment and the therapist needs to fill it quickly, the appointment monitoring system 112 can share the notification to the highest-ranked users first, those who are statistically most likely to accept and attend. The AI engine also provides a corresponding explanation for each ranking for every user in the list, providing a clear and interpretable reason why they were assigned that particular position in the ranking. For example, an explanation says, “User has a 90% predicted attendance rate when it comes to fill-ups”. Another explanation might state, “User ranked lower due to multiple last-minute cancellations and lack of response to recent fill-in offers.” Beneficially, in a therapy clinic, the waitlist managing environment 100 could be used to optimize how open slots are filled, ensuring that the most committed users receive priority scheduling.

In operation 218, a confirmation module 120 confirms the booking of the appointment schedule after the user submits the user's selection. The confirmation module 120 plays a role in finalizing the appointment scheduling process, and in at least one embodiment, the confirmation module 120 performs critical tasks in finalizing the appointment scheduling process. Upon the user's submission of their selection for a preferred at least one session slot, the confirmation module 120 initiates the confirmation procedure. The primary function of the confirmation module 120 is to validate and confirm the chosen at least one session slot, ensuring that the user's selection aligns with the available slots and meets the specified preferences. This confirmation step acts as a crucial checkpoint to prevent errors or discrepancies in the appointment scheduling process, safeguarding against potential misunderstandings or double bookings. By providing users with a definitive confirmation of their chosen appointment schedule through emails, and messages on the user device 110, the confirmation module 120 enhances transparency and instils confidence in the user.

The appointment monitoring system 112 further includes a contextual analysis module 122 configured to interpret details entered by the user as at least one user preference. The contextual analysis module 122 uses LLM (Large Language Model) to interpret details entered by the user as at least one user's preferences.

The contextual analysis module 122 is operatively coupled to the appointment monitoring system 112 and plays a crucial role in enhancing the user experience. The contextual analysis module 122 is designed to interpret the details provided by users during the appointment scheduling process and translate them into specific user preferences. Using advanced technology, such as the LLM (Large Language Model), the contextual analysis module 122 employs sophisticated algorithms to comprehend the nuances of user input accurately. For instance, it can distinguish preferences regarding appointment timings, specific therapists, or recurring sessions from the information provided by the user. By effectively interpreting user preferences, the contextual analysis module 122 ensures that the appointment scheduling platform 102 can provide individual needs with precision and efficiency. This capability not only streamlines the scheduling process but also contributes to a more personalized and user-centric experience, ultimately enhancing satisfaction levels for both users and service

The waitlist managing environment 100 transforms the appointment scheduling platform 102 by efficiently managing waitlists in real-time, ensuring optimal utilization of available slots. Employing machine learning algorithms to prioritize users and customizing notifications based on individual preferences, the waitlist managing environment 100 enhances user experience and maximizes appointment fulfilment. The contextual analysis module further improves accuracy in understanding user preferences, ultimately streamlining the scheduling process and reducing wait times, thus benefiting both users and service providers alike.

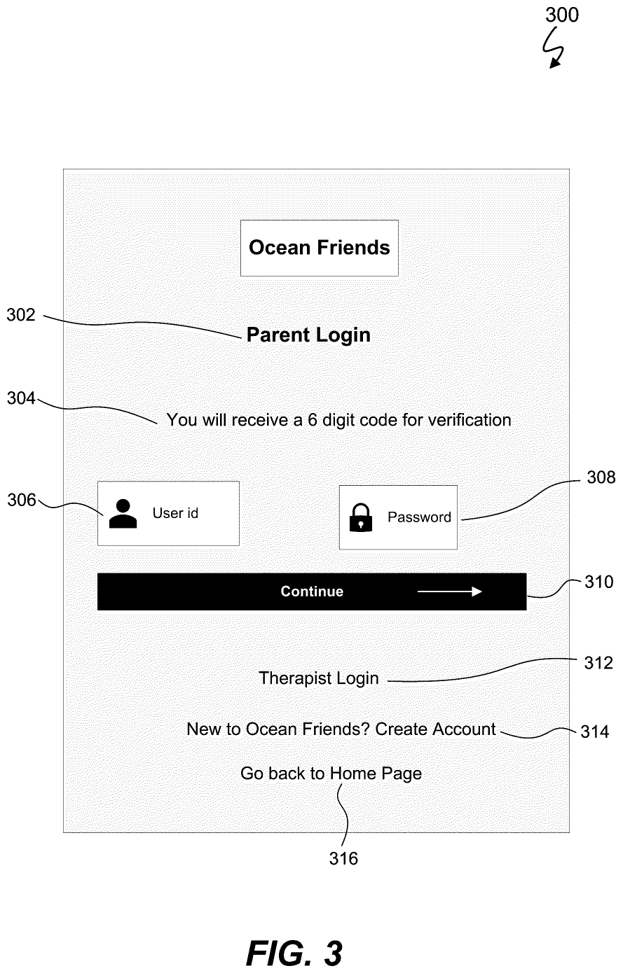

FIG. 3 depicts an exemplary view of the login page 300 of the appointment scheduling platform 102.

The exemplary login page 300 of the appointment scheduling platform 102 is disclosed here, which can be accessed by the user who has the login credentials of the appointment scheduling platform 102. In this exemplary scenario, an example is considered where the user is the parent of the child, who is undergoing some therapy. The patient can be anyone who has direct access to the appointment scheduling platform 102. Further, the user can access the appointment scheduling platform 102 using his/her device, i.e., a user device 110, which may include a mobile, tablet, laptop, computer, or any other similar device.

The login page 300 of the appointment scheduling platform 102 includes tab 302 ‘Parent Login’ representing the title of the login page 300 and tab 304 ‘You will receive a 6-digit code for verification’ which represents that the user upon entering the logging details will receive the 6-digit code on his/her connected device for verification. The verification code may be received through an SMS, WhatsApp message, email, and so on in user device 110.

Tabs 306 and 308 represent ‘User ID’ and ‘Password’ respectively, entering which the user clicks on tab 310 ‘Continue’. Also, there is tab 312 ‘Therapist Login’ clicking on which the therapist can log in to the appointment scheduling platform 102 and may check his/her scheduled appointments and make the changes if needed.

If the user is a new user to the appointment scheduling platform 102, the user can create a new account by clicking on tab 314 ‘Create a new account’. Further, if the user wishes to go back to the homepage of the appointment scheduling platform 102, the user can click on tab 316 ‘Homepage’.

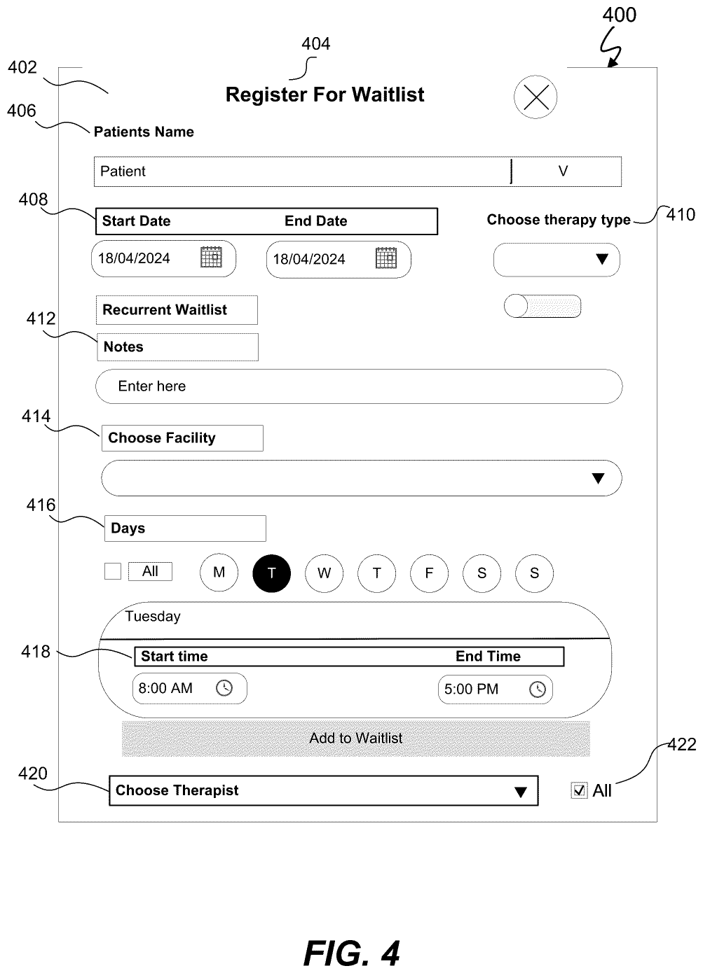

FIG. 4 depicts an exemplary view of the user interface 400 disclosing form 402 where users can fill in the details to register for the waitlist.

The exemplary form 402 disclosed in the user interface 400 of the appointment scheduling platform 102 is shown in FIG. 4. Form 402 includes user preferences like day, date, time, therapy type, location, choice of therapist, and so on to get registered on the waitlist. The form 402 includes tab 404 ‘Register for Waitlist’, which denotes the title for form 402. Form 402 allows users to enter their preferences based on which the notifications are provided to the user, whenever there is any cancellation or change in the schedule of the session slots.

Form 402 further includes tab 406 ‘Patient's Name’ where the user can enter his/her name. Further, using tab 408 ‘Start Date’ and ‘End Date’, the user can enter the dates till the user needs therapy sessions. The user here can mention both the starting end for the therapy and the end date for the therapy. For example, if a user has missed an appointment and he is registering for the waitlist, he needs the therapy session to be conducted within 2 weeks because the next therapy session is scheduled for the 3rd week. So, in this case, the user will mention the start date as the beginning of the 1^{st} week and the end date as the end of the 2^{nd} week.

Further, using tab 410 ‘Choose Therapy Type’ the user can choose the type of therapy which they want to opt for the patient using a drop-down menu. The therapy type could be a single therapy or a group of therapies that the patient undergoes. For example, it may include options like OT, PT, ST; OT, PT; ST; PT, ST, and so on. Here OT, PT, and ST stand for occupational therapy, physical therapy, and speech therapy respectively. This is just an exemplary scenario, although there could be any number of options depending upon the therapies, in the case of the present example, available in that therapy center. Tab 412 ‘Notes’ allows the user to enter the preferences that are not mentioned by default in form 412. The contextual analysis module 122 takes the intent behind the user preferences and based on that the machine learning module 114 generates some recommendations for the user.

The user can fill in other preferences like facility, days, time slots, therapist, and so on by filling in the details on various tabs like tab 414 ‘Choose Facility’, ‘tab 416 ‘Days’, tab 418 ‘Start Time’ and ‘End Time’. The user can either select at least one preferred therapist from the drop-down list present in tab 420 ‘Choose Therapist’ or may directly select tab 422 ‘All’ which will directly select all the therapists belonging to that particular therapy. Similarly, the user may select the date on which they want to schedule the therapy by clicking on tab 416 ‘Day’ and choosing the preferred date. The user may even choose different dates or the same dates for at least one therapy session, at their convenience.

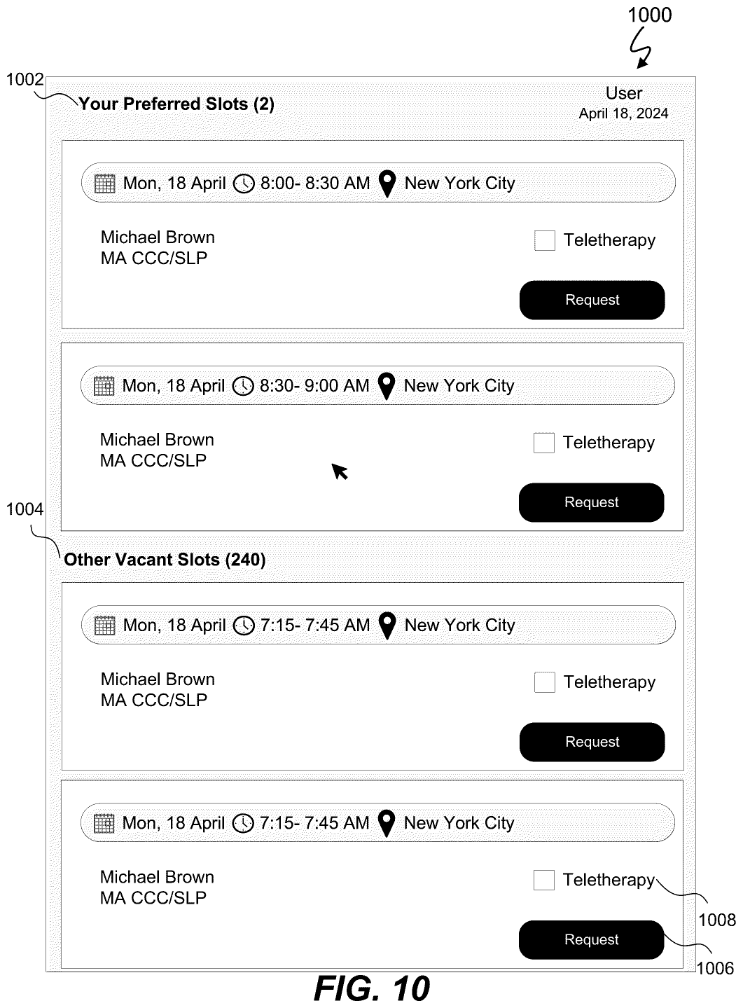

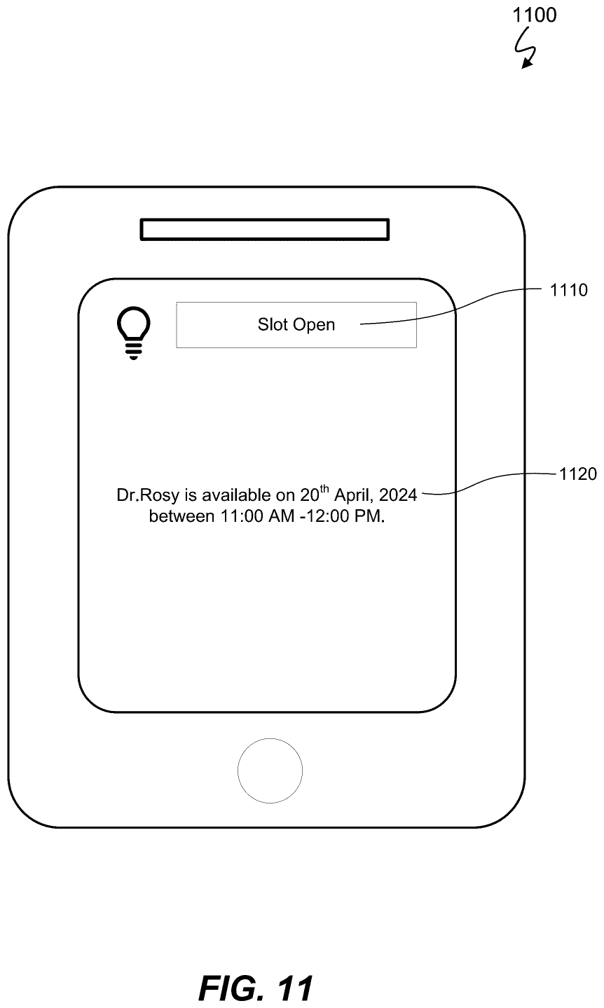

Finally, after finally after filling in all the preferences, the user may click on tab 424 ‘Add to Waitlist’, and submit his/her preferences based on these preferences the machine learning module 114 and contextual analysis module 122 will analyze the at least one preferences provided by the user and pick up the best match of the session slots for the user. The notification module 118 further notifies the at least one match to the user which the user can select according to his/her choice and availability. This is discussed in detail in the latter section in FIGS. 10 and 11.

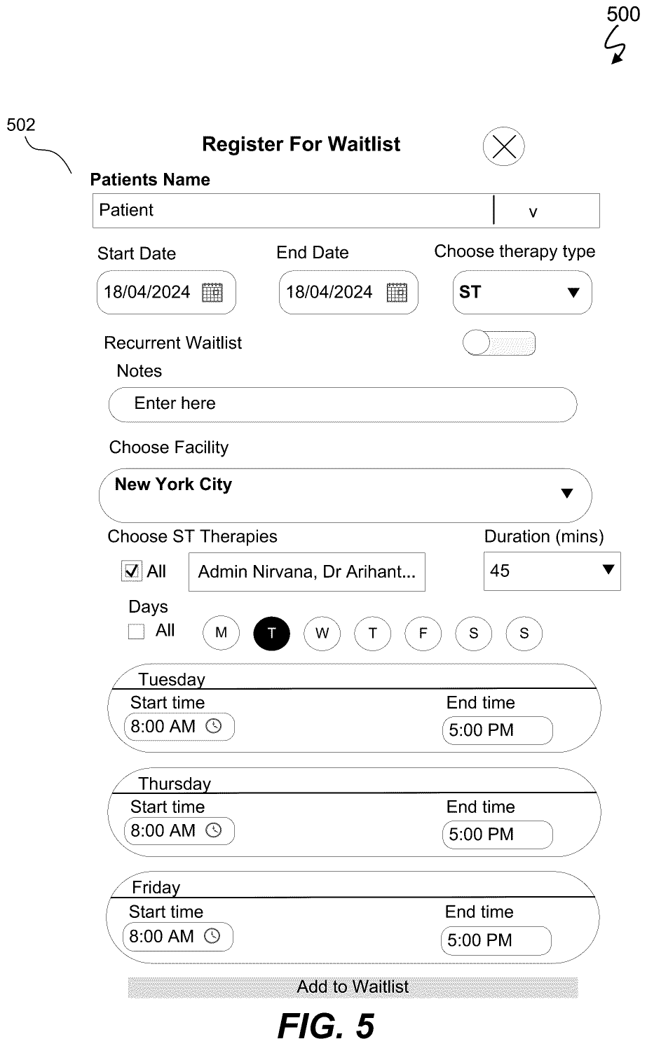

FIG. 5 depicts an exemplary view of the user interface 500 disclosing a form 502 filled out by the user to register for the waitlist.

The exemplary form 502 disclosed in the user interface 500 of the appointment scheduling platform 102 is shown in FIG. 5. The user interface 500 shows form 502 which is filled up by the user to register the patient on the waitlist. Form 502 is a filled-up version of form 402. All the details like patient name, day, date, time, location, therapy type, therapist name, and so on are filled up by the user.

Based on these details of the preference of the user, the machine learning module 114 and contextual analysis module 122 will analyze the at least one preference provided by the user and pick up the best match of the session slots for the user. The notification module 118 further notifies the at least one match to the user, which the user can select according to his/her choice and availability. This is discussed in detail in the latter section in FIGS. 10 and 11.

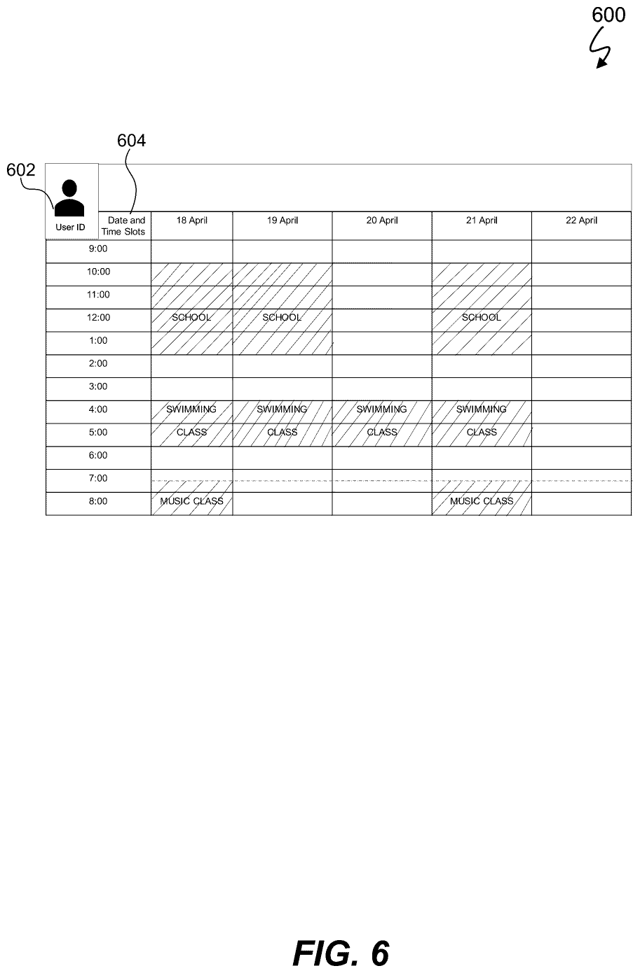

FIG. 6 depicts an exemplary view of schedule 600 stored in appointment scheduling platform 102.

The exemplary view of the patient's schedule 600 is disclosed herein, which is provided by the user, i.e., the parent of a child, in the present example. The patient's schedule 600 is stored in memory 106, which is integrated within the appointment scheduling platform 102. The user and the staff of the therapy center (present example) can both access the patient's schedule 600 by clicking on the corresponding tab available on the appointment scheduling platform 102. This is just an exemplary scenario showing the patient's schedule 600, besides this, the various other details of the patient are also made available on the appointment scheduling platform like medical history, treatment history, basic details like name, age, address, email, and so on.

Patient schedule 600 includes a tab ‘User Id’ 602, where the name and other details of the user are mentioned. Here in this example, the user is the mother of the child undergoing therapy. It further includes ‘Date’ 604 and ‘Time Slots’, which depict the child's daily schedule. For example, the child remains busy from ‘10:00 AM-1:00 PM’ daily, except on ‘20 Oct. 2023’ because of his school. Similarly, if we talk about a single day, say ‘18 Oct. 2023’, the child remains busy between ‘10:00 AM-1:00 PM; 4:00 AM-5:00 PM; and 7:00 AM-8:00 PM’ So, in this case, the contextual analysis module 122 will analyze the intent behind the data provided by the user and based on that machine learning module 114 will provide at least one recommendations of at least one available appointment schedules when the user is free.

Based on the inputs provided by the user, the machine learning module 114 and contextual analysis module 122 will generate at least one recommendation of available appointment schedules. If the presented recommendations are as per user preferences, the user may book them or else have another option to opt for the waiting list option, where the new available at least one session slots will be made available to the user based on his/her preferences.

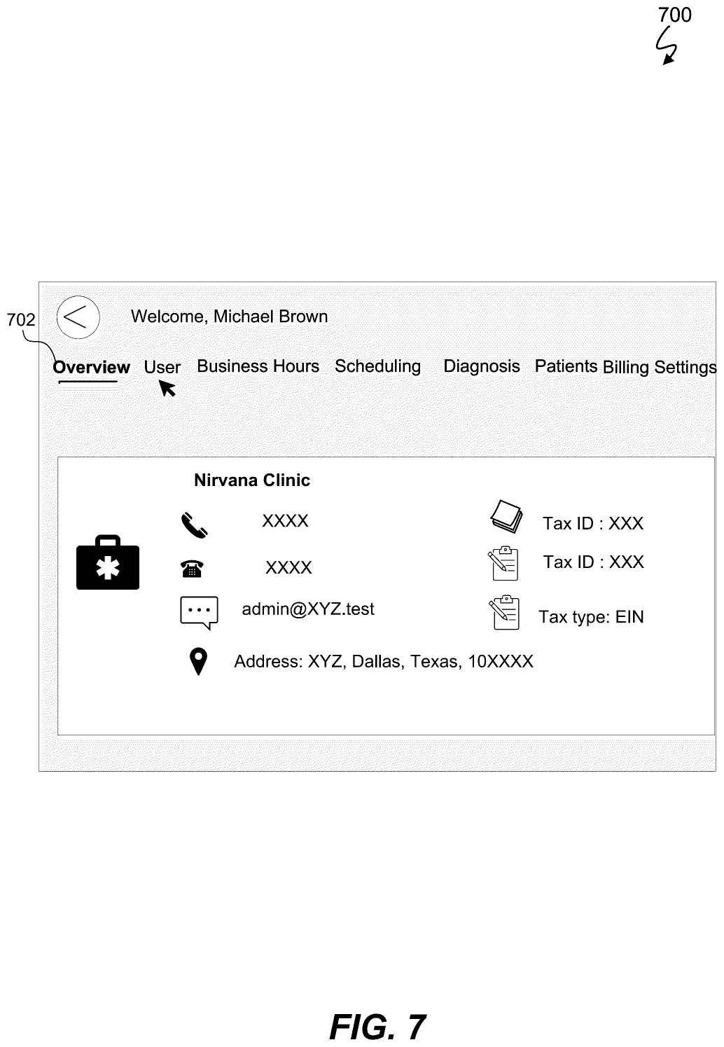

FIG. 7 depicts an exemplary view of the user interface 700 showing the details of the therapy center chosen as per at least one user preference.

The user interface 700 shows the details of the therapy center chosen by the users by providing at least one preferences when the user clicks on tab 702 ‘Overview’. Details like the name of the clinic, contact details of the clinic, address of the clinic, email ID of the clinic, and so on are displayed on the user interface 700 so that the user can easily have access to the details of the clinic while using the appointment scheduling platform 102.

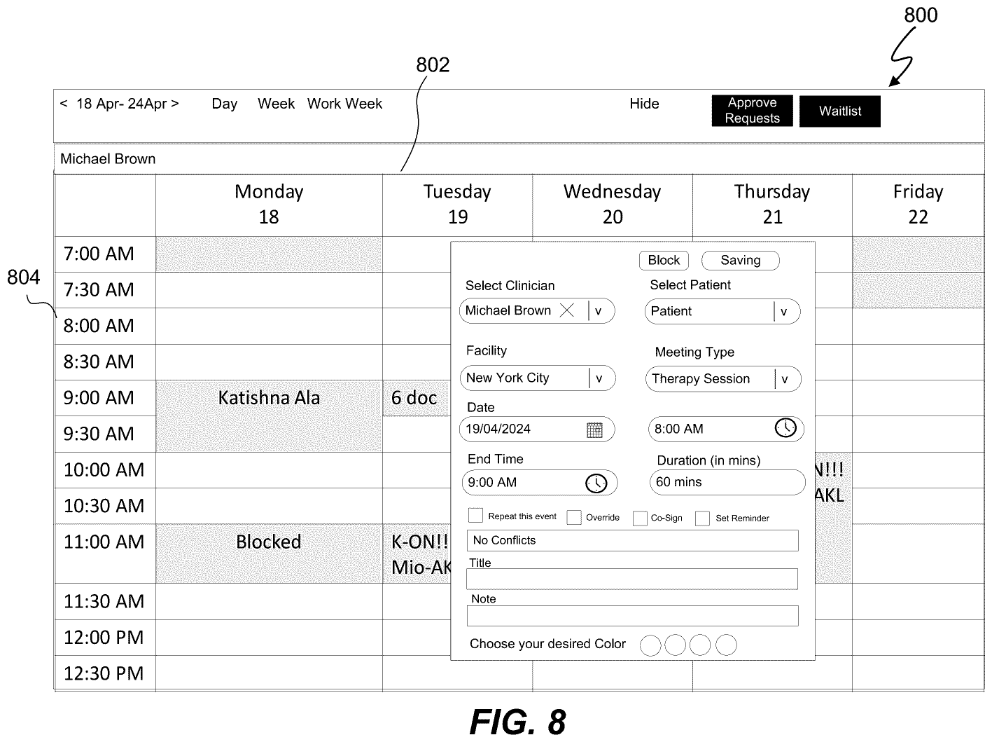

FIG. 8 depicts an exemplary view of the user interface 800 showing the calendar view of the booked and vacant appointment schedule.

The user interface 800 shows the calendar view of the booked and vacant appointment schedule. The places shown with dark colors depict that they are already booked by some user and the places shown with white color depict that they are still vacant and any user can book the session slot of that time interval. The calendar displayed herein includes ‘Date’ 802 mentioned in columns and ‘Time’ 804 mentioned in rows.

The appointment managing environment 100 provides access to the therapist to make changes in the booked session slots in case of any emergency or any other situation. For example, if the therapist has an emergency and he has an appointment booked with a child for behavioral therapy, he may not be able to conduct as he has to leave the clinic before that time. So, in that case, the therapist may reschedule the booked session slot of the user, and based on the changes made, the user will be notified that the appointment is rescheduled. If the user is comfortable with that session slot, the user can confirm and book that session slot, or else the user can opt for the waitlist option to wait for the session slot of his/her choice.

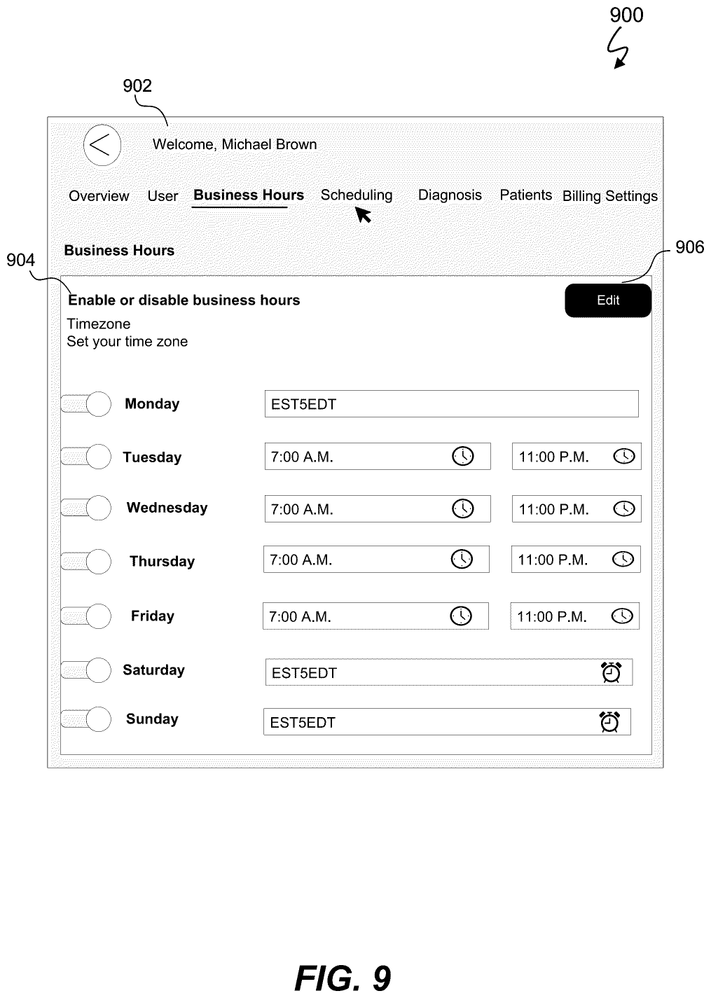

FIG. 9 depicts an exemplary view of user interface 900 showing the working hours of the expert.

By clicking the tab 902 ‘Business Hours’ on the user interface 900, the working hours of the therapist are displayed. The therapist can enable or disable the working hours by clicking on tab 904. The therapist can also edit his/her working hours by clicking on tab 906 ‘Edit’.

FIG. 10 depicts an exemplary view of the user interface 1000 showing at least one preferred slot and vacant session slots in the appointment scheduling platform 102.

The user interface 100 uses the machine learning module 114 and contextual analysis module 122 and presents a set of at least one ‘Preferred Slots’ 1002 and at least one ‘Other Vacant Slots’ 1004 to the user on the appointment scheduling platform 102. The user can select any of the displayed slots that best suit his/her preference just by clicking on the tab 1006 ‘Request’ placed in front of every session slot. The displayed session slots include details like the name of the therapist, qualification, and specialization of the therapist, day, date, time at which the therapist is available for the therapy sessions, location of the clinic, and so on. Further, the user can also opt for teletherapy service by clicking on tab 1008 ‘Teletherapy’.

FIG. 11 depicts an exemplary view of the notification 1100 shared to the user device 110 when the new session slots are available for booking.

The exemplary view of the user device 110, which is a mobile device 1100, in the case of the present example, is disclosed herein. In this example, the user is scheduling an appointment for his/her child. The user defined in this example is the parent of the child. In the present example, an appointment is scheduled for the child, who is the patient in this case and is undergoing some therapies in a therapy center.

The mobile device 1100 of the user disclosed herein is not only limited to a mobile device but may also include a tablet, laptop, computer, and so on. Similarly, the notification 1120 shared with the user on his/her mobile device 1100 may be obtained via. any medium like SMS, WhatsApp, Telegram, email, or any other similar platform, and so on.

The user device i.e., a mobile device includes a notification heading named “Slot Open!!” 1110 and a message 1120 which states:

“Dr. Rosy is available on 19 Apr. 2024 between 11:00 AM-12:00 PM.

Click on the link given below to book the appointment before somebody else does”.

This is just an exemplary scenario. A notification message 1120 for a similar kind is shared with the user whosoever has opted for the waiting list option.

In this case, for example, the mother of the child waited to book at least one preferred session slots, who is undergoing therapies at a therapy center. The mother has some fixed preferences for the therapist and timings at which the child is available to attend the therapy sessions. The machine learning module 114 has generated some recommendations of available appointment schedules for her child based on her preferences. However, she was not comfortable with the timings or therapist of the recommended appointment schedule and as a result, she selected the waiting list option, which allows her to wait for the availability of the preferred session slot. There might be some situations like another patient being absent, someone cancelling the appointment, and so on, which results in the availability of new session slots for scheduling appointments. Then in this case, the user who has opted for the waiting list option will be notified along with a share link to book the appointment, if the newly available session slot fits their requirements.

However, this is just an exemplary scenario, where the user did not make any bookings and directly chose the waiting list option. There may be scenarios where the user is not satisfied with the recommendation of at least one available appointment schedules provided to him/her but still, as they don't have any other option besides this and looking after the best option at the current moment, they may schedule an appointment that fits best in that situation and further opt for waiting list option as well, so that in the case in future if any cancellation is there and at least one preferred session slots is available which fulfils his/her preference, then at that time the user may select the preferred session slot and cancel the old booking.

The appointment managing environment 100 provides notifications to all those users who have opted for the waiting list option. There are some criteria based on which the users are selected from the waiting list which may include first come first serve basis, revenue-based, emergency cases, internal ratings allotted to each user using contextual model, users whose insurance companies pay the amount on time, users who respond on time, and so on.

The user gets notified from the message 1120 shared with them and the user can book the slots directly using the given link or by using the appointment scheduling platform.

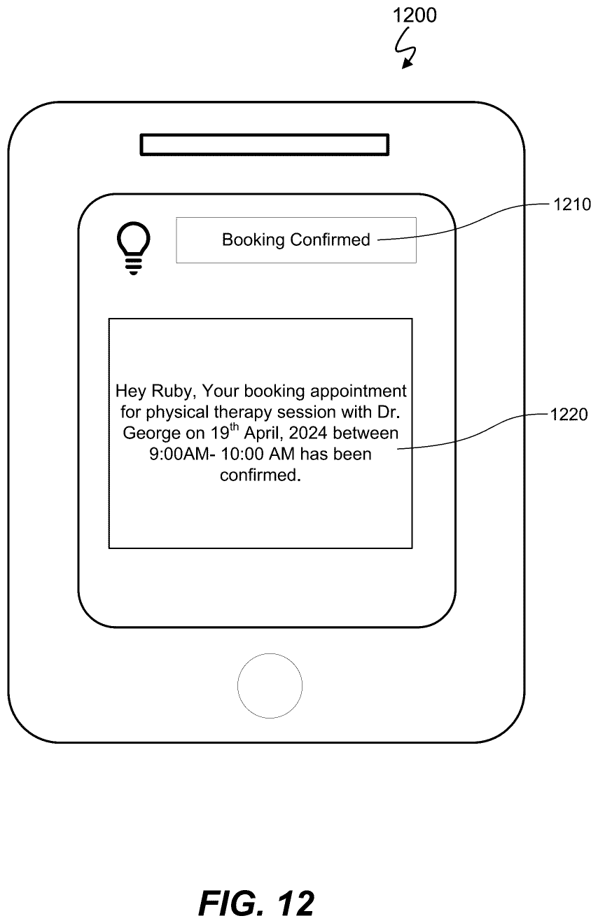

FIG. 12 depicts an exemplary view of the notification i.e., confirmation message shared to the user device 110, when the appointment scheduled by the user is confirmed.

The exemplary view of the user device 110 which is a mobile device 1200, in the case of the present example, is disclosed herein. In this example, the user is scheduling an appointment for his/her child i.e., the user defined in this example is the parent of the child.

The mobile device 1200 of the user disclosed herein is not only limited to a mobile device but may also include a tablet, laptop, computer, and so on. Similarly, the notification 1220 shared with the user on his/her mobile device 1200 may be obtained via. any medium like SMS, WhatsApp, Telegram, email, or any other similar platform, and so on.

The user device i.e., a mobile device includes a notification heading named “Booking Confirmed!!” 1210 and a message 1220 which states:

“Hey Ruby, your booking appointment for a Physical therapy session with Dr. George on 19 Apr. 2024 between 9:00 AM-10:00 AM has been confirmed”.

This is just an exemplary scenario, a notification message 1220 of a similar kind is shared with the user once the booking is confirmed by the user. There may be scenarios where the user wishes to make multiple bookings on their preferred at least one session slots but they do not get the required session slots because they are unavailable, in that case, they may either wait for the corresponding session slot availability or they are choosing some other appointment schedule which fits best at that situation. Further, there may be scenarios in which the user may opt for the waitlist option and book an appointment of their preferred session slot using that waitlist option.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive like the term “comprising” as “comprising” is interpreted when employed as a transitional word in a claim.

### Technical Advancements:

The appointment scheduling platform manages a waitlist by prioritizing the user based on the user, such as a patient, preferences, and real-time availability of vacant slots has several technical advantages, including, but not limited to, the realization of:
guided and constrained use of artificial intelligence in the appointment monitoring system 112;
allows users to personalize appointments in real-time;
optimizes the allocation of appointment slots, ensuring that the healthcare provider's time is utilized effectively and efficiently, leading to improved productivity;
provides a mechanism of waiting list for users to express interest in preferred slots that are currently unavailable;
provides flexibility to users while selecting preferences;
user friendly;
easy to use;
versatility of the user interface;
provides notifications to at least one users whenever there is any change in the status of the slots;
generates valuable insights into appointment trends, user preferences, and scheduling patterns, which in turn leads to improved service and patient outcomes;
quick access to therapy services and reduced waiting time;
does not require manual intervention;
maintains transparency and security of the data.

The embodiments herein and the various features and advantageous details thereof are explained concerning the non-limiting embodiments in the following description. Descriptions of well-known components and processing techniques are omitted to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. Those skilled in the art will recognize that the embodiments herein can be practiced with modification within the spirit and scope of the embodiments as described herein.

Unless the context indicates otherwise, throughout the specification and claims which follow, the word “comprises” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context dictates otherwise.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in at least one embodiments.

As used in this specification and the appended claims, the singular forms “a,” “an,” and “the” include plural referents unless the content dictates otherwise. It should also be noted that the term “or” is generally employed in its broadest sense, that is, as meaning “and/or” unless the content dictates otherwise.

The use of the expression “at least” or “at least one” suggests the use of at least one elements or ingredients or quantities, as the use may be in the embodiment of the disclosure to achieve at least one of the desired objects or results.

Any discussion of documents, acts, materials, devices, articles, or the like that has been included in this specification is solely to provide a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions, or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

We claim:

1. A method for managing a waitlist in an appointment scheduling platform comprising:
executing code by a data processing system to perform operations comprising:
receiving a request from a user to book an appointment schedule based on at least one user preference;
identifying at least one available appointment schedule corresponding to the at least one user preferences, wherein each appointment schedule includes at least one session slot;
generating a prompt to import user preferences and guide and constrain an Artificial Intelligence (AI) engine to perform operations comprising:
operate on multiple scheduling factors of waitlisted users to present a waitlist option to the user through a user interface if the at least one user preference does not match with the available appointment schedules, wherein the user provides at least one waitlist related information via the presented user interface; and
identify at least one preferred vacant session slot in real-time based on the provided at least one waitlist related information and cancellation of session slots by other users; and
notifying the user about the availability of the at least one preferred vacant session slots, wherein the notification is sent to the user based on at least one pre-defined criterion.

2. The method of claim 1, wherein the at least one user preference includes at least one expert's detail, preferred date and time of the therapy session, and overall therapy duration.

3. The method of claim 1, wherein the at least one user preference comprises a number of therapy sessions already available in a given time duration, a time required to visit the therapy center, an appointment type, a therapy type, an insurance eligibility, and user responsiveness.

4. The method of claim 1, wherein the at least one pre-defined criterion further comprises exclusion criteria to decide whether the user is to be excluded from sharing the notifications about the availability of at least one vacant session slots.

5. The method of claim 4, wherein the exclusion criteria include exclusion of the user if the user responds late to the shared notifications, has a high cancellation rate, does not make insurance payments on time, and is located distantly from the therapy center.

6. The method of claim 1, wherein generating the prompt for the AI engine comprises incorporating:
a session start date and a session end date defining the appointment schedule;
a user request for the appointment schedule;
historical attendance data of the user, including past session count, cancellation count, and no-show count; prior fill-in appointment history associated with each user;
at least one therapist-generated note per user; and
optional metadata, including waitlist notes provided by the user.

7. The method of claim 1, wherein the notification shared with the user includes an access link to view available vacant session slots and an option to book at least one vacant slot.

8. The method of claim 1, wherein the time duration between notifications shared with the user can be customized by the expert.

9. The method of claim 1, wherein the AI engine is guided and constrained to notify the user about the availability of the at least one preferred vacant session based on candidate-specific data comprising: a count of past attended sessions, a count of past cancellations and no-shows, a record of prior fill-in participation, at least one therapist-provided notes, and supplemental waitlist notes associated with the user.

10. The method of claim 1, wherein the AI engine is guided and constrained to return a structured JSON output comprising a list of users ranked by predicted attendance and a corresponding explanation for each ranking.

11. A data processing system for managing a waitlist in an appointment scheduling platform that can be accessed by a user using a user device, the data processing system comprising:
at least one processors; and
a memory, coupled to the one more processors, storing code that when executed by the at least one processors causes the data processing system to perform operations comprising:
receive a request from a user to book an appointment schedule based on at least one user preferences, wherein the at least one user preferences include at least one expert's details, timestamps, date of therapy session, and session duration;
identify at least one available appointment schedules corresponding to at least one user preferences, wherein each appointment schedule includes at least one session slots; and
collect at least one requests from the user to be added to a waitlist if at least one user preferences do not match with at least one available appointment schedules;
an appointment monitoring system to track the waitlist in case of at least one appointment cancellations comprises:
a machine learning module to generate a prompt to import user preferences and guide and constrain an artificial intelligence engine to perform operations comprising to operate on multiple scheduling factors of waitlisted users to prioritize the users on the waitlist based on a plurality of factors; and
a comparator to match at least one user preferences to create an eligibility list of the user, wherein the eligibility list is created based upon the exclusion criteria which decides who all users are to be excluded from sharing the notifications about the availability of at least one vacant session slots;
a notification module to notify the user of the eligibility list upon the availability of at least one vacant session slots due to at least one cancelled appointment based on machine learning algorithm-based prioritization of the user;
a confirmation module to confirm the booking of the appointment schedule after the user submits the user selection.

12. The system of claim 11, further includes a contextual analysis module configured to interpret details entered by the user as at least one user preference.

13. The contextual analysis module of claim 12, uses at least one Large Language Models (LLMs) to interpret details entered by the user as at least one user preference.

14. A non-transitory, computer program product for monitoring video of a meeting room for distracted participants, the computer program product having executable code stored therein that when executed by at least one processors causes a computer system to perform operations comprising:
receiving a request from a user to book an appointment schedule based on at least one user preference;
identifying at least one available appointment schedule corresponding to the at least one user preferences, wherein each appointment schedule includes at least one session slot;
generating a prompt to import user preferences and guide and constrain an artificial intelligence engine to perform operations comprising:
operate on multiple scheduling factors of waitlisted users to present a waitlist option to the user through a user interface if the at least one user preference does not match with the available appointment schedules, wherein the user provides at least one waitlist related information via the presented user interface; and
identify at least one preferred vacant session slot in real-time based on the provided at least one waitlist related information and cancellation of session slots by other users; and
notifying the user about the availability of at least one preferred vacant session slot, wherein the notification is sent to the user based on at least one pre-defined criterion.

15. The non-transitory, computer program product of claim 14, wherein the at least one user preference includes at least one expert's detail, preferred date and time of the therapy session, and overall therapy duration.

16. The non-transitory, computer program product of claim 14, wherein the at least one user preference comprises a number of therapy sessions already available in a given time duration, a time required to visit the therapy center, an appointment type, a therapy type, an insurance eligibility, and user responsiveness.

17. The non-transitory, computer program product of claim 14, wherein the at least one pre-defined criterion further comprises exclusion criteria to decide whether the user is to be excluded from sharing the notifications about the availability of at least one vacant session slots.

18. The non-transitory, computer program product of claim 14, wherein the exclusion criteria include exclusion of the user if the user responds late to the shared notifications, has a high cancellation rate, does not make insurance payments on time, and is located distantly from the therapy center.

19. The non-transitory, computer program product of claim 14, wherein the notification shared with the user includes an access link to view available vacant session slots and option to book at least one vacant slot.

20. The non-transitory, computer program product of claim 14, wherein the time duration between notifications shared with the user can be customized by the expert.

21. The non-transitory, computer program product of claim 14, wherein generating the prompt for the AI engine comprises incorporating:
a session start date and a session end date defining the appointment schedule;
a user request for the appointment schedule;
historical attendance data of the user, including past session count, cancellation count, and no-show count; prior fill-in appointment history associated with each user;
at least one therapist-generated notes per user; and optional metadata, including waitlist notes provided by the user.

22. The non-transitory, computer program product of claim 14, wherein the AI engine is guided and constrained to notify the user about the availability of the at least one preferred vacant session based on candidate-specific data comprising: a count of past attended sessions, a count of past cancellations and no-shows, a record of prior fill-in participation, at least one therapist-provided notes, and supplemental waitlist notes associated with the user profile.

23. The non-transitory, computer program product of claim 14, wherein the AI engine is guided and constrained to return a structured JSON output comprising a list of users ranked by predicted attendance and a corresponding explanation for each ranking.
